(19) United States
(12) Patent Application Publication
(10) Pub. No.: US 2021/0287806 A1
(43) Pub. Date: Sep. 16, 2021

(54) DEVICES AND METHODS OF REMOTELY MONITORING AND TRIAGING PATIENTS

(71) Applicant: AIRx Health, Inc., San Jose, CA (US)

(72) Inventors: Vijaykumar R. RAJASEKHAR, Campbell, CA (US); Marlon Sebastian CASTILLO, San Mateo, CA (US); Frank James BUNCOM, IV, San Diego, CA (US)

(21) Appl. No.: 17/003,809

(22) Filed: Aug. 26, 2020

Related U.S. Application Data
(60) Provisional application No. 62/989,492, filed on Mar. 13, 2020.

(51) Int. Cl. G16H 50/30; G16H 40/67; A61B 5/0205; A61B 5/00

(52) U.S. Cl. CPC G16H 50/30; A61B 5/0022; A61B 5/02055; G16H 40/67

(57) ABSTRACT

A method for remotely monitoring one or more patients may include receiving a transmission of one or more patient assessments of current health of each patient, determining a trending index for each patient based on a baseline medical risk of the patient, a social exposure risk of the patient, and at least one patient assessment for the patient; and triaging the one or more patients according to medical risk associated with the contagious disease based on the trending index. The patient assessment may, for example, include an evaluation of at least one of: at least one symptom associated with a contagious disease and at least one vital sign associated with the contagious disease. The method and associated systems may be used, for example, to monitor and/or triage patients in the outpatient setting, to help manage patients who are at medical risk for a contagious disease.

### CROSS-REFERENCE TO RELATED APPLICATIONS

This application claims priority to U.S. Patent Application Ser. No. 62/989,492 filed Mar. 13, 2020, which is incorporated herein in its entirety by this reference.

### TECHNICAL FIELD

This invention relates generally to the field of medical treatment of illnesses, and more specifically to devices and methods for remotely monitoring and outpatient triaging patients with contagious diseases.

### BACKGROUND

When contagious (e.g., viral) illnesses spread rapidly within a region, healthcare resources in the region are likely be stretched, resulting in limits of medical treatment for patients. For example, as COVID-19 has spread within the U.S., guidelines from the Centers for Disease Control and Prevention (CDC) recommend patients with “flu-like” symptoms (fever/cough/dyspnea) to contact their local doctor. Unfortunately, it is not always easy to contact one's primary doctor, especially during a time when an exponential number of patients may simultaneously feel symptomatic and seek care. For example, phone lines may become overloaded and while patients are alternatively instructed to go to the emergency room. However, emergency rooms quickly become overwhelmed, and there are not enough ICU isolation units. As a result, many patients are unable to receive the medical attention and treatment that are required. Accordingly, there is a need for new and improved devices and methods to address these shortcomings.

### SUMMARY

In some variations, a method for remotely monitoring one or more patients may include, at one or more processors, receiving a transmission of one or more patient assessments of current health of each patient, determining a trending index for each patient based on a baseline medical risk of the patient, a social exposure risk of the patient, and at least one patient assessment for the patient; and triaging the one or more patients according to medical risk associated with the contagious disease based on the trending index. Each patient assessment may include, for example, an evaluation of at least one of: at least one symptom associated with a contagious disease and at least one vital sign associated with the contagious disease. The method may be used to remotely monitor a variety of patients, such as patients awaiting results of a test for the contagious disease, patients who have tested positive for the contagious disease, and/or patients associated with a person who has tested positive for the contagious disease (or suspected to be positive for the contagious disease).

Specifics of the patient assessment may vary depending on the type of contagious disease for which patients are being monitored. In some variations, the contagious disease may be a viral disease. The viral disease may, for example, affect the respiratory system. For example, the viral disease may be caused by a coronavirus (e.g., associated with Middle East Respiratory Syndrome (MERS), Severe Acute Respiratory Syndrome (SARS), etc.), an influenza virus, etc. In one example variation, the contagious disease for which the patients are being monitored is COVID-19 (SARS-CoV-2). In some variations, the patient assessment may include an evaluation of at least one symptom selected from the group consisting of: fever, cough, dyspnea, fatigue, chills, myalgia, rhinorrhea, sore throat, nausea, vomiting, headache, abdominal pain, diarrhea, hemoptysis, chest congestion, and loss of smell and/or taste. Additionally or alternatively, the patient assessment may include an evaluation of at least one symptom selected from the group consisting of new onset chest pain, new onset back pain, and new onset walking difficulty. Furthermore, in some variations the patient assessment may include an evaluation of breathing difficulty during one or more specified activities. In some variations, the evaluation of at least one symptom may include a severity rating on a numerical scale.

Furthermore, in some variations the patient assessment may include an evaluation of at least one vital sign selected from the group consisting of resting oxygen saturation, and oxygen saturation immediately following mild activity (e.g., exercise), or both. For example, in some variations the patient assessment may additionally or alternatively include an evaluation the difference between oxygen saturation immediately following mild activity, and oxygen saturation measured at rest. The patient assessment may additionally or alternatively include other suitable vital signs, such as temperature and/or heart rate.

In some variations, the trending index may be based on an aggregation (e.g., sum) of a first risk score associated with baseline medical risk for the patient, a second risk score associated with social exposure risk for the patient, and a third risk score associated with at least one patient assessment for the patient. The first risk score may, for example, be quantified based on one or more characteristics selected from the group consisting of: age, sex, pregnancy, presence of a chronic medical condition, engagement in a chronic health-adverse behavior, and prescription of a specified medication.

The second risk score may, for example, be quantified based on one or more characteristics selected from the group consisting of: occupation as a healthcare worker, previous presence in a healthcare facility, previous presence in close proximity situation with other people, previous positive test for the contagious disease, association with a person who is suspected or confirmed as tested positive for the contagious disease, and association with a person exhibiting symptoms of the contagious disease. In some variations, the second risk score may be further quantified based on the patient having close contact with a person having one or more characteristics selected from the group consisting of: occupation as a healthcare worker (or essential worker or other occupation in a high-risk (e.g., public) environment), previous presence in a healthcare facility, previous presence in close proximity situation with other people, positive test for the contagious disease, a pending test result for the contagious disease, association with a person who is suspected or confirmed as tested positive for the contagious disease, and association with a person exhibiting symptoms of the contagious disease.

In some variations, the third risk score may be quantified based at least in part on severity of a symptom or vital sign in a most recent patient assessment. Additionally or alternatively, the third risk score may be quantified based on change in severity of a symptom or vital sign between the most recent patient assessment and at least one prior patient assessment. In some variations, two separate risk scores may be quantified in the above scores as included as separate additional risk scores for inclusion in the trending index.

Furthermore, in some variations determining the trending index for each patient may further include aggregating a fourth risk score associated with number of days since onset of at least one symptom associated with the contagious disease.

A patient may be prompted to complete a patient assessment at programmed intervals (e.g., daily). In some variations, at least one patient may be prompted via a SMS text message comprising a hyperlink to an interactive online survey. Additionally or alternatively, at least one patient may be prompted over an automated phone system with at least one of a programmable voice service and a programmable text service. Furthermore, in some variations the method may include transmitting a programmed reminder to at least one patient to complete the patient assessment, in response to failing to receive the patient assessment from the at least one patient by a predetermined time. Additionally or alternatively, the method may include transmitting a programmed reminder to at least one designated contact of a patient in response to failing to receive the patient assessment from the patient by a predetermined time.

The method may further include communicating to one or more medical care practitioners patient data associated with the one or more patients. The communicated patient data may include at least one selected from the group consisting of: at least one patient assessment, at least one trending index, and the results of triaging the one or more patients according to medical risk associated with the contagious disease. Additionally or alternatively, the method may include communicating a programmed alert for at least one patient characterized as having a threshold medical risk associated with the contagious disease.

### BRIEF DESCRIPTION OF THE DRAWINGS

FIG. 1A is a schematic illustration of an example variation of a remote monitoring system.

FIG. 1B is a schematic illustration of an example variation of a medical risk characterization system in a remote monitoring system.

FIG. 2 is a schematic flowchart of an example variation of method for remotely monitoring and/or triaging patients in relation to a contagious disease.

FIG. 3 depicts an example variation of a graphical user interface (GUI) through which a healthcare institution may invite a patient to participate in remote monitoring.

FIG. 4A depicts an example variation of a GUI through which a patient may enter account information during patient enrollment with a remote monitoring system.

FIG. 4B depicts an example variation of a GUI requesting patient consent during patient enrollment with a remote monitoring system.

FIG. 5 is a schematic illustration of a portion of an example variation of a method for remotely monitoring and/or triaging patients in relation to a contagious disease.

FIG. 6 depicts an example variation of a communication (SMS test message) to direct a patient to complete a patient assessment.

FIG. 7 depicts an example variation of a GUI providing an introduction to the online survey

FIGS. 8A-8C depict example variations of GUIs prompting the patient to provide evaluations of vital signs associated with COVID-19.

FIGS. 9A-9C depict example variations of GUIs prompting the patient to provide evaluations of symptoms associated with COVID-19.

FIG. 10 depicts an example variation of a GUI confirming transmission of a patient assessment.

FIGS. 11A and 11B are schematic illustrations of a portion of an example variation of a method for remotely monitoring and/or triaging patients in relation to a contagious disease.

FIG. 12 is a list of exemplary prompts for use in a patient assessment in an exemplary variation of a method of remotely monitoring and triaging patients.

FIG. 13 is an exemplary graphical user interface providing a summary report for a group of remotely monitored patients.

FIG. 14 is an exemplary graphical user interface providing a patient report for a remotely monitored patient.

### DETAILED DESCRIPTION

Non-limiting examples of various aspects and variations of the invention are described herein and illustrated in the accompanying drawings.

Various methods and systems for remotely monitoring and/or outpatient triaging patients with illnesses are described herein. Such methods and systems may, for example, be used to help monitor and manage patients who are at risk for contagious diseases, such as viral diseases.

For example, in view of the COVID-19 pandemic, health systems need an easy way to manage patients who are infected with or are otherwise at risk for COVID-19. For patients with COVID-19, symptoms such as fever and/or cough typically progress to breathing difficulty over time (e.g., on average, about 7 days after onset of symptoms). During this period, home isolation or quarantine may be typical, with patient symptoms, vital signs, etc. tracked to determine whether and when to safely escalate care of these patients (e.g., hospitalization). Similarly, persons who are suspected to have been exposed to someone with COVID-19 (e.g., close contact with someone who has tested positive for COVID-19 or is presumptive positive for COVID-19, who have arrived from a region with a high occurrence of COVID-19 within the population, etc.) may also be instructed to engage in home isolation or quarantine.

The methods and systems described herein may be used to remotely monitor and triage patients in this and similar scenarios, such as for health systems and regional health departments to manage infectious outbreaks. For example, the methods and systems described herein may be used to remotely monitor patients who have tested positive for a contagious disease, patients who are awaiting results of a test for an contagious disease, patients who are closely associated with (e.g., have close contact with, such as household contact or workplace contact) a person who has tested positive for the contagious disease or is otherwise suspected to potentially have the contagious disease, and/or patients who are recovering from the contagious disease (e.g., discharged from hospital). Patients under home monitoring/surveillance can be monitored and, if warranted, tested for the contagious disease, without going into traditional healthcare facilities where others could be exposed to the disease. Furthermore, those assessed to be at lower risk can continue to self-monitor at home, while those assessed with escalating changes in symptoms can be pre-identified to get hospital attention sooner. Patients with other contagious diseases (e.g., other viral diseases such as other coronavirus diseases (e.g., caused by viruses associated with Middle East Respiratory Syndrome (MERS), Severe Acute Respiratory Syndrome (SARS), etc.), influenza, etc.) may similarly be monitored and/or managed.

Advantageously, the methods and systems described herein may allow medical teams to monitor their patients and assist with either home management or escalation of care, and/or provide alerts and/or other information to governmental health officials. The system may be programmed to automatically triage patients by risk level through a medically adopted protocol to be used by local medical teams to monitor and triage patients in the outpatient setting. By monitoring and triaging patients in the outpatient setting, this may advantageously relieve load on hospitals, clinics, and other in-person medical care institutions and free such institutions for more severe and critical cases requiring direct treatment. Accordingly, the devices and methods such as those described herein for remote monitoring and triaging may significantly increase the ability for healthcare systems to manage outbreaks of illnesses. Furthermore, the methods and systems described herein are designed to be convenient and easy-to-use, thereby improving patient compliance and improving the ability to accurately and promptly identify patients whose escalating illnesses warrant medical attention.

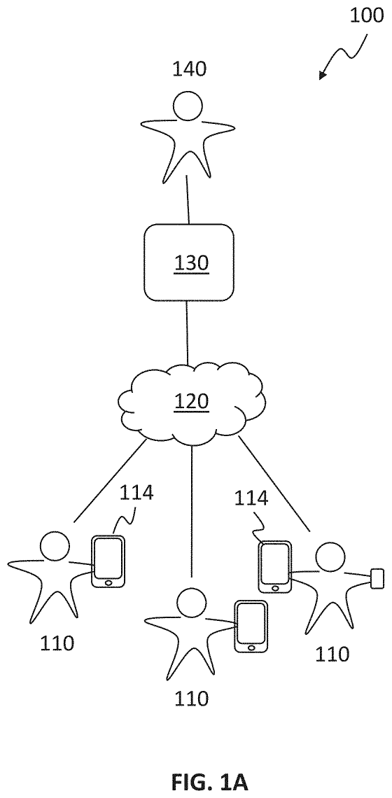

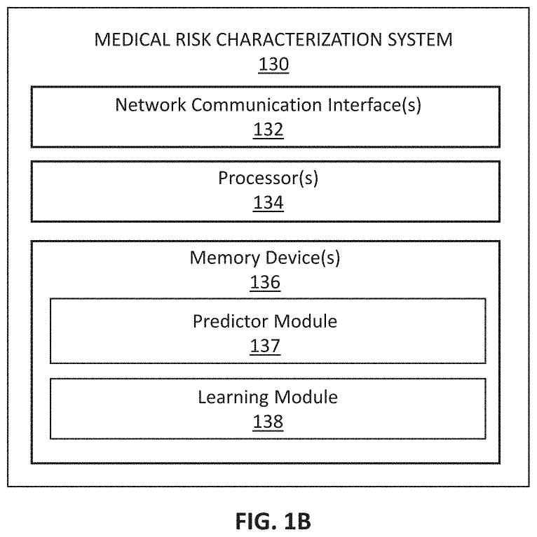

As shown in FIG. 1A, in some variations, a remote monitoring system 100 may interact with a plurality of patients 110. Each patient may be enrolled with the system to verify contact information and other patient information, obtain appropriate patient consents, etc. as described in further detail below. Each patient 110 may have or be associated with (e.g., via a family member or other contact) a user computing device 114 that may be used to communicate patient data over one or more networks 120 to a medical risk characterization system 130. Patient data may be transmitted wirelessly, for example (e.g., through Bluetooth, WiFi, 3G/4G/5G cellular networks, etc.) and in a HIPAA-compliant manner. As shown in FIG. 1B, a medical risk characterization system 130 may include at least one network communication interface 132 for receiving patient data, at least one processor 134, and at least one memory device 136 for storing received patient data. Furthermore, the one or more memory devices may be configured to store one or more software modules for analyzing the received and stored bodily metrics. For example, the memory device(s) 132 may include a predictor module 137 configured to assess patients' medical risk for a contagious disease based on patient data. Additionally or alternatively, the memory device(s) 136 may include a learning module 138 configured to train and/or apply a machine learning model for assessing medical risk for a contagious disease, as further described below.

As described in further detail below, the patient data may be provided through a reporting protocol performed by the patient as part of a regular (e.g., daily) assessment and include, for example, information relating to symptoms, vital signs, social exposure risk (e.g., activities that may increase the risk profile of the patient with respect to the contagious disease), and/or any other suitable information. Such patient data may be processed by the medical risk characterization system 130 to triage the one or more patients according to medical risk associated with the contagious disease based on the trending index, thereby identifying those patients who may be in more need of urgent medical attention. As described in further detail below, the patient data, the results of triaging, and/or the identities of patients in most need of urgent medical attention, etc. may be communicated to one or more medical care providers 140 who may act accordingly (e.g., contact patients, provide medical guidance or treatment, etc.). Patient data may additionally or alternatively be collected into patient reports and/or uploaded into suitable electronic health records. In some variations, the system 100 may be associated with a virtual clinic staffed by physicians and/or other medical care professionals. Additionally or alternatively, the system 100 may be associated with non-virtual (e.g., “brick-and-mortar”) medical institutions. Medical care teams at the virtual clinic and/or non-virtual medical institutions may use the system 100 to monitor patients and can “order” tests, prescribe or adjust medication and/or medical treatment (e.g., for a contagious disease associated with respiratory conditions, treatment with supplemental oxygen may be prescribed or adjusted), and/or provide other suitable medical guidance (e.g., bed rest), etc. as medically relevant and when available.

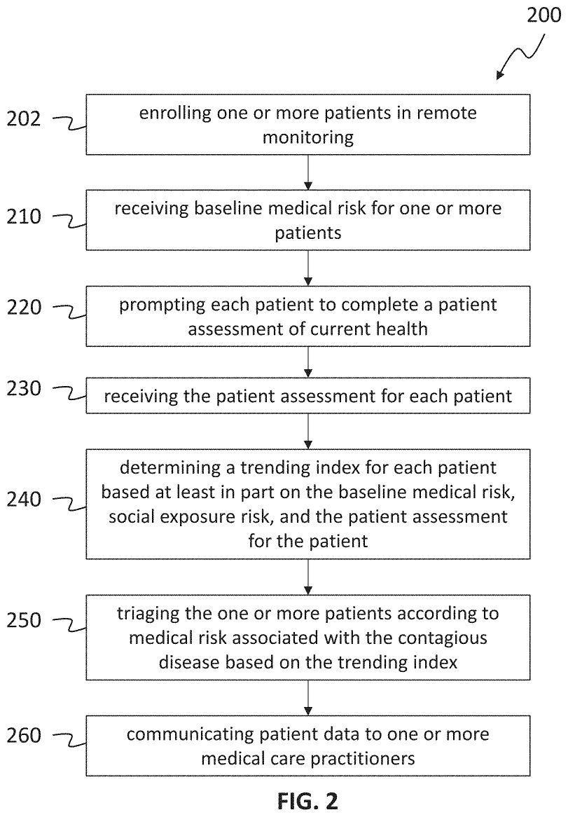

FIG. 2 illustrates an example variation of a method 200 for remotely monitoring one or more patients. Method 200 may include, for example, enrolling one or more patients in remote monitoring 202, receiving baseline risk for one or more patients 210, prompting each patient to complete a patient assessment of current health 220 (e.g., symptoms, vital signs, etc.), and receiving the patient assessment for each patient 229. Patient data, including the baseline risk and patient assessment, may be received in various suitable manners as described below. After receiving the patient assessment(s), the method may further include characterizing or assessing medical risk for the patient, particularly with respect to the contagious disease. For example, in some variations, the method 200 may include determining a trending index 240 based at least in part on the baseline medical risk, social exposure risk, and the patient assessment for the patient, triaging the one or more patients 250 according to medical risk associated with the contagious disease based on the trending index, and/or communicating patient data 260 (e.g., baseline risk information, social exposure risk, patient assessment(s), etc.) to one or more medical care practitioners. Further details of the methods are described below.

### Patient Enrollment

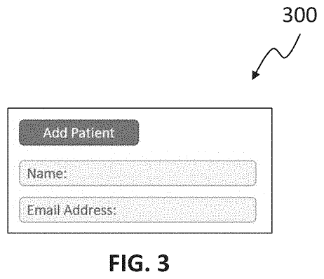

Patients may be selected or invited by a healthcare institution (e.g., medical care provider) to participate in remote monitoring. For example, FIG. 3 illustrates an example graphical user interface 300 (GUI) through which a healthcare institution may invite a patient to participate in remote monitoring. GUI 300 may be displayed, for example, on a suitable user computing device that is accessible by the healthcare institution. GUI 300 includes, for example, fields for entering a patient name and patient contact information (e.g., email address). Once these fields are completed, the indicated patient may be added to the system for invitation (e.g., selection of “Add Patient” button), and the system may contact the patient via the patient contact information to complete patient enrollment with the remote monitoring system. Although GUI 300 displays fields for receiving patient name and email address, it should be understood that GUI 300 may additionally or alternatively include other suitable patient-specific information used for initiating patient enrollment. For example, GUI 300 may include a patient medical record number, phone number, and/or other patient identification information. Furthermore, in some variations at least a portion of GUI 300 may be embedded in a pre-existing user interface in a third party software program (e.g., electronic medical record system) by way of an application-specific interface (API) that may enable interoperability of the remote monitoring system 100 with a third party software program. For example, an electronic medical record system may incorporate an “Add patient to remote monitoring” button that interfaces with the remote monitoring system with a suitable API, etc. Accordingly, this may enable the remote monitoring system to directly pull or receive suitable patient information from the patient's medical record, and subsequently invite the patient to perform additional steps as described below to complete enrollment. Additionally or alternatively, the API may enable the remote monitoring system to push suitable patient information (e.g., data such as patient assessments including symptoms and/or vital signs) to the patient's medical record in an electronic medical record system.

Once contacted by the remote monitoring system, a patient may complete an online or otherwise electronic form that is available through a suitable website, portal, mobile application, and the like. Through this form, the patient (or representative such as a family member, friend, medical care provider, etc.) may provide patient information (e.g., contact information, baseline risk information, social exposure risk information, an initial patient assessment of symptoms and/or vital signs, etc.) and/or complete one or more suitable telehealth consent forms. The information may then be reviewed by a licensed care provider to prescribe or order remote monitoring for the illness of interest (e.g., “flu-like” symptoms).

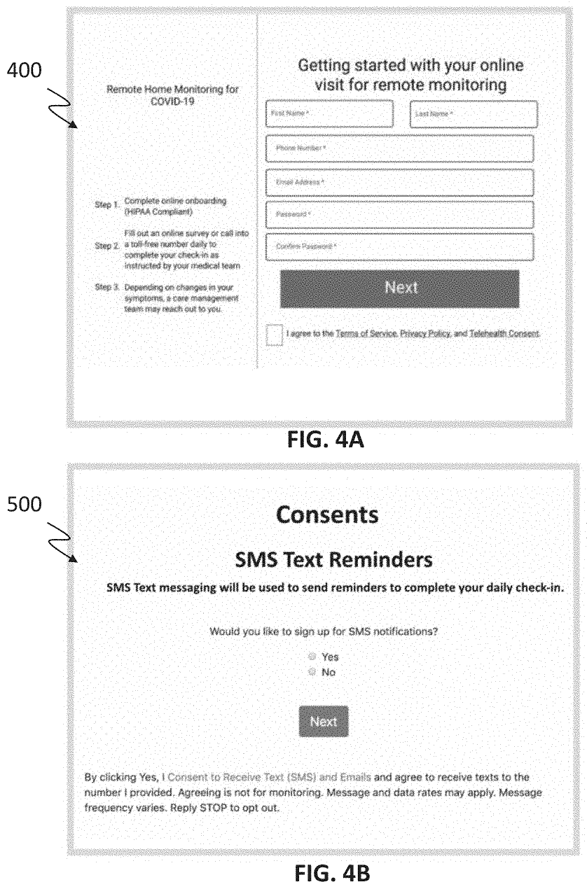

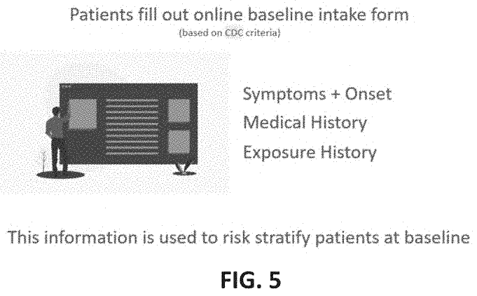

For example, a patient may receive an electronic message (e.g., email, SMS text message, etc.) with a hyperlink that, once selected, directs the patient to a patient account set-up form. FIG. 4A illustrates an example GUI 400 through which a patient may enter account information such as patient name, phone number, email address, and password for use in establishing an account with the remote monitoring system. As described above, the patient may further be prompted to complete one or more suitable consent forms. For example, FIG. 4B illustrates an example GUI 500 that requests the patient to consent to receiving SMS text reminders to complete a daily patient assessment (“daily check-in”).

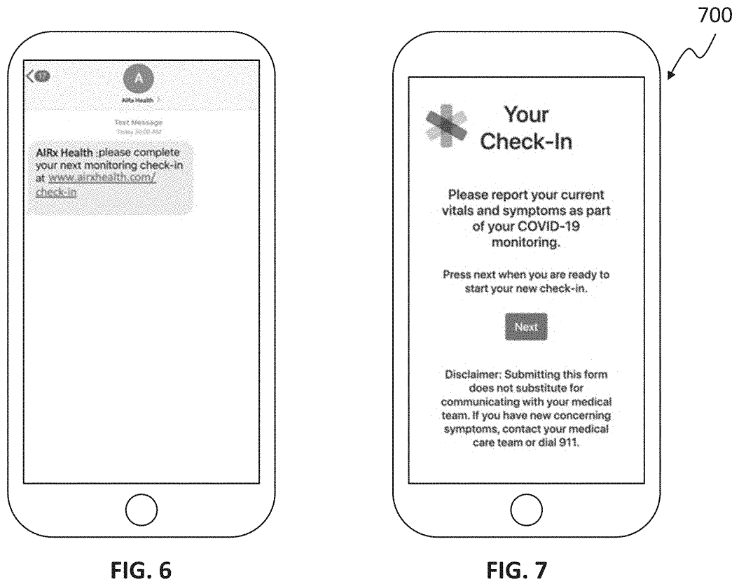

Additionally, as shown in FIG. 5, a patient enrolling in remote monitoring may provide baseline risk information (e.g., medical history) and social exposure risk information, which may be used to perform medical risk stratification across monitored patients at baseline. Baseline risk information may, for example, include demographics (e.g., age, sex, etc.), medical history (e.g., chronic medical conditions and other underlying conditions or comorbidities, prescription of medications), engagement in a health-adverse behavior (e.g., smoking). The requested information may be specific to the particular contagious disease(s) of interest with respect to that patient. For example, for a patient being monitored with respect to COVID-19, baseline risk information may include evaluation of chronic lung disease(s) (e.g., COPD, asthma, or other lung disease), diabetes mellitus, cardiovascular disease (e.g., congestive heart failure (CHF), acute coronary syndrome (ACS), hypertension (HTN), arrhythmia, stroke, or other cardiovascular disease), chronic renal disease, chronic liver disease, other immunocompromised condition, neurological or neurodevelopmental disability, other intellectual disability, obesity, and/or other chronic disease. Additionally or alternatively, baseline risk information may include indication of whether the patient is pregnant or smokes. Furthermore, baseline risk information for monitoring COVID-19 may include an indication whether the patient takes certain medications such as an ACE inhibitor, angiotensin II receptor blockers (ARBs), and/or anti-inflammatory medications (e.g., NSAIDs, cortisone, etc.).

Furthermore, a patient enrolling in remote monitoring may provide social exposure risk information (e.g., travel history, known or potential contact with others who are ill or potentially ill, etc.), etc. Social exposure risk information for the patient may include, for example, occupation as a healthcare worker, previous presence in a healthcare facility, previous presence in close proximity situation with other people, previous positive test for the contagious disease, association with a person who is suspected or confirmed as tested positive for the contagious disease, and association with a person exhibiting symptoms of the contagious disease, etc. Additionally or alternatively, social exposure risk information may include whether the patient has had close contact with (e.g., lives with, works with, or otherwise had has close contact with) anyone who would be able to indicate occupation as a healthcare worker, previous presence in a healthcare facility, previous presence in close proximity situation with other people, previous positive test for the contagious disease, association with a person who is suspected or confirmed as tested positive for the contagious disease, and association with a person exhibiting symptoms of the contagious disease, etc. In some variations, appropriate consents may be obtained to enable use of this social exposure risk information to assist in contact tracing efforts to investigate and/or further mitigate outbreaks of the contagious disease.

An initial patient assessment may further be provided during patient enrollment. For example, the initial patient assessment may include an evaluation of presence and/or severity of one or more symptoms, date/time of onset of such symptoms, evaluation of one or more vital signs, etc.

In some variations, patient enrollment information may be obtained through an online form. Additionally or alternatively, patient enrollment information may be obtained over an automated phone interview (e.g., over an automated phone system with programmable voice and/or programmable text services). For example, patient enrollment may be performed in a manner similar to that described in U.S. patent application Ser. Nos. 16/664,434 and/or 62/955,223, each of which is hereby incorporated in its entirety by this reference.

In some variations, at least some information provided during patient enrollment may be used to automatically complete or partially complete other standard health forms, such as CDC's PUI form, thereby improving efficiency in providing crucial information to healthcare officials during events such as an outbreak.

Patient enrollment may also include providing the patient with one or more monitoring equipment such as a thermometer and/or pulse oximeter (e.g., if the patient does not already have one) for subsequent use in remote monitoring. Alternatively, patients may utilize personal equipment. Exemplary devices for use in monitoring and triaging systems and methods described herein as described in further detail in U.S. patent application Ser. Nos. 16/664,434 and/or 62/955,223 (incorporated by reference above).

### Patient Monitoring

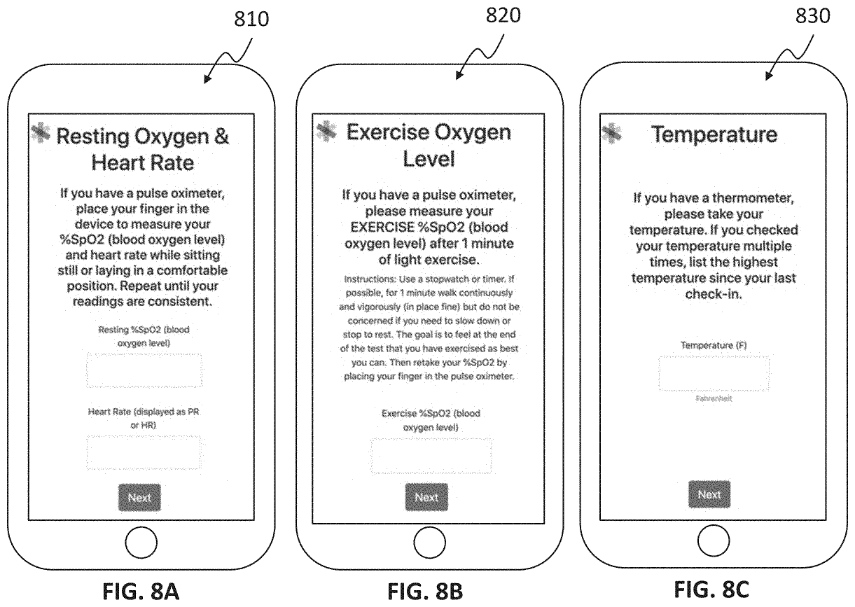

Patient data may be received in one or more suitable manners. For example, in some variations, the remote monitoring system may include an interactive survey that may prompt a patient (or a user associated with the patient) to complete and submit a patient assessment. The survey may, for example, be an online survey accessible over the Internet, a survey integrated in a mobile application executed in a user computing device (e.g., smartphone or tablet), or the like. In some variations, as shown in FIG. 6, the remote monitoring system may communicate a message (e.g., SMS test message, email message, or other notification, etc.) to the patient that includes a hyperlink to the online survey such as, a link to a suitable user interface in the mobile application for completing the patient assessment, etc. The message may be regularly communicated (e.g., daily, twice daily, etc.) as a programmed reminder for the patient to complete a scheduled patient assessment, and/or may be communicated to the patient. In some variations, the frequency of the programmed reminder may vary with previous assessments of severity of medical risk for the patient so as to enable closer monitoring or tracking of patients considered to be a greater medical risk. For example, patients considered as at greater medical risk for the contagious disease, such as evaluated using a triaging protocol as described below, may receive more frequent reminders to complete a patient assessment. Additionally or alternatively, a programmed reminder may be communicated to the patient in response to failing to receive the patient assessment by a predetermined time (e.g., after 2 hours following a first programmed reminder, or before a predetermined time each day). For example, the system may send an SMS text message to the patient with a hyperlink at one or more scheduled times each day for the patient to log in and complete the online survey, such as “Please complete your assessment by clicking this link.” Furthermore, a programmed reminder may be communicated to designated contact (e.g., designated care supporter such as a family member, friend, household contact of the patient, medical care provider for the patient, etc.) in response to failing to receive the patient assessment by a predetermined time.

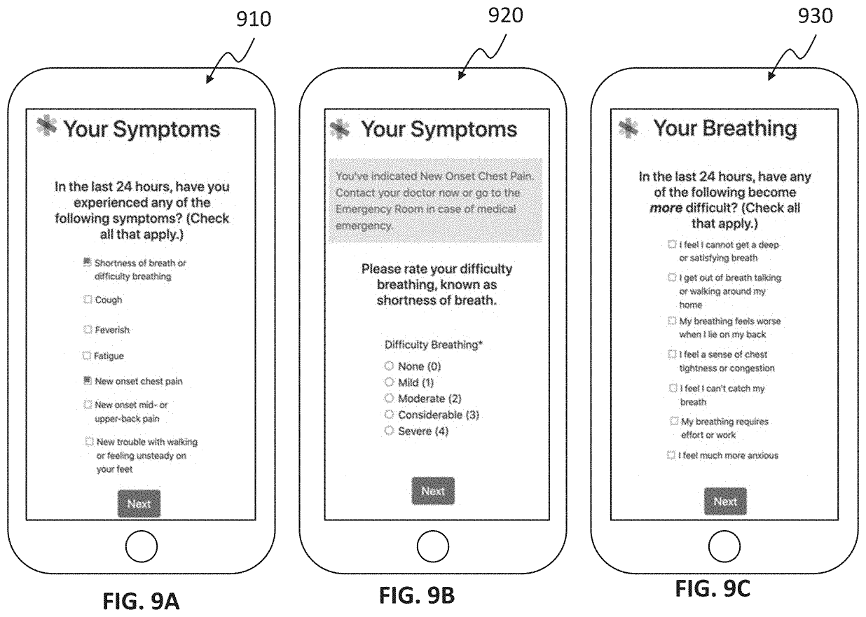

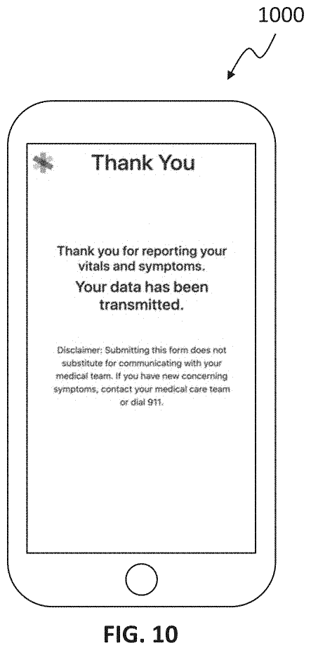

Accordingly, to complete a patient assessment, the patient may complete an online survey. FIG. 7 illustrates an example GUI 700 providing an introduction to the online survey, including a brief explanation of the content and/or purpose of the online survey. The survey may systematically prompt the patient to answer one or more questions relating to at least one vital sign and/or at least one symptom associated with the contagious disease for which the patient is being monitored. For example, FIGS. 8A-8C depict example GUIs prompting the patient to provide evaluations of vital signs associated with COVID-19. Specifically, FIG. 8A illustrates an example GUI 810 prompting the patient to provide blood oxygenation level (% SpO₂) and heart rate measured by the patient while at rest (e.g., siting still, lying in a comfortable position). GUI 810 may further include instructions to the patient describing how to appropriately measure these vital signs (e.g., repeat until measurements are consistent), which may improve the accuracy of patient assessments and the resulting triaging efforts by the systems. FIG. 8B illustrates an example GUI 820 prompting the patient to provide a blood oxygenation level measured by the patient following mild activity (exercise % SpO₂) such as mild exercise (e.g., walking continuously for one minute). Like GUI 810, GUI 820 may include instructions to the patient how to appropriately measure these vital signs (e.g., describe nature and duration of suitable mild exercise), which may help to improve consistency of conditions for measuring exercise % SpO₂ (e.g., between multiple patient assessments for a patient, across different patients, etc.). Furthermore, FIG. 8C illustrates an example GUI 830 prompting the patient to provide a temperature (e.g., to evaluate presence of fever), along with other suitable instructions such as to provide the highest temperature measured since the patient's more recent patient assessment.

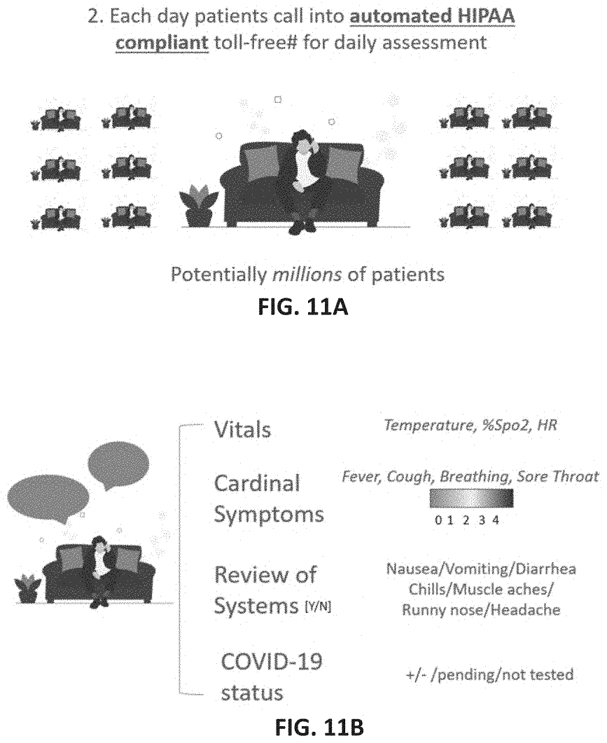

In some variations, the survey may further prompt the patient to answer one or more questions relating to at least one symptom associated with the contagious disease for which the patient is being monitored. For example, FIGS. 9A-9C depict example GUIs prompting the patient to provide evaluations of symptoms associated with COVID-19. FIG. 9A illustrates an example GUI 910 providing the patient with a list of symptoms associated with COVID-19, including shortness of breath of difficulty breathing, cough, feverishness, fatigue, new onset chest pain, new onset mid- or upper-back pain, and new trouble with walking or feeling unsteady on his or her feet.

In some variations, the system may further prompt the patient to indicate severity of one or more of the indicated symptoms. For example, as shown in FIG. 9B illustrating example GUI 920, the system may prompt the patient to indicate severity of a symptom as a dynamic follow-up question (e.g., following selection of “shortness of breath” on GUI 910, the patient may be prompted via a subsequent GUI to indicate severity of shortness of breath). GUI 920 prompts the patient to characterize the severity of his or her shortness of breath on a qualitative scale, but in other variations GUI 920 may similarly prompt the patient to characterize the severity of his or her shortness of breath on a quantitative scale, such as between 0 and 4. Additionally or alternatively, the system may prompt the patient to indicate severity at the same time as indicating presence of a system (e.g., the GUI 910 may include an entry field or series of buttons corresponding to symptom severity). As described in further detail below, other suitable dynamic questions may also be asked in follow-up to a provided response (e.g., the system may prompt certain questions to the patient conditioned upon certain previous responses by the patient).

It should be understood that additional GUIs may be included in the online survey to gather any changes or updates to other patient data, such as baseline medical risk information (e.g., new chronic medical conditions or medications, or indication that the patient is newly pregnant) and/or social exposure risk (e.g., recent visits to hospital, test results, close contact exposure to other person(s) at risk for the contagious disease, etc.). At least some information may additionally or alternatively be automatically pulled from the patient's medical record for consideration by the remote monitoring system (e.g., new positive test for the contagious disease).

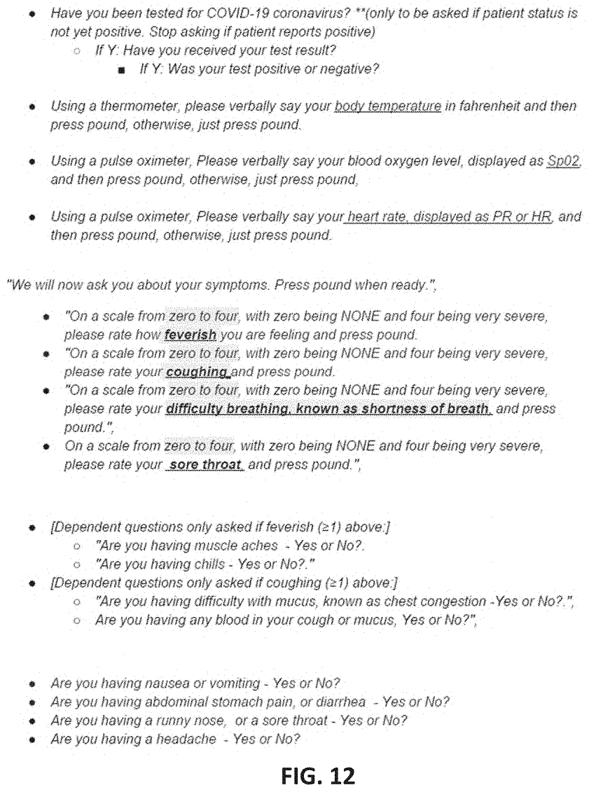

Once the online survey is completed, the survey may provide a confirmation screen (e.g., GUI 1000 as shown in FIG. 10) confirming transmission of the patient assessment, as well as a reminder to contact a medical care professional if appropriate.

While FIGS. 7, 8A-8C, and 9A-9C illustrate an example variation of an online survey intended to receive a patient assessment, in some variations the system may receive a patient assessment through an automated phone system. For example, in some variations the remote monitoring system 100 may include an automated phone system (e.g., accessibly by dialing a toll-free number, text messaging a designated number, etc.) for patients with illness-specific symptoms to report their daily vitals and symptoms to healthcare teams. The automated phone system may, for example, include a programmable voice service and/or a programmable text service, and may include methods and systems similar to that described in further detail in U.S. patent application Ser. Nos. 16/664,434 and/or 62/955,223 (incorporated by reference above).

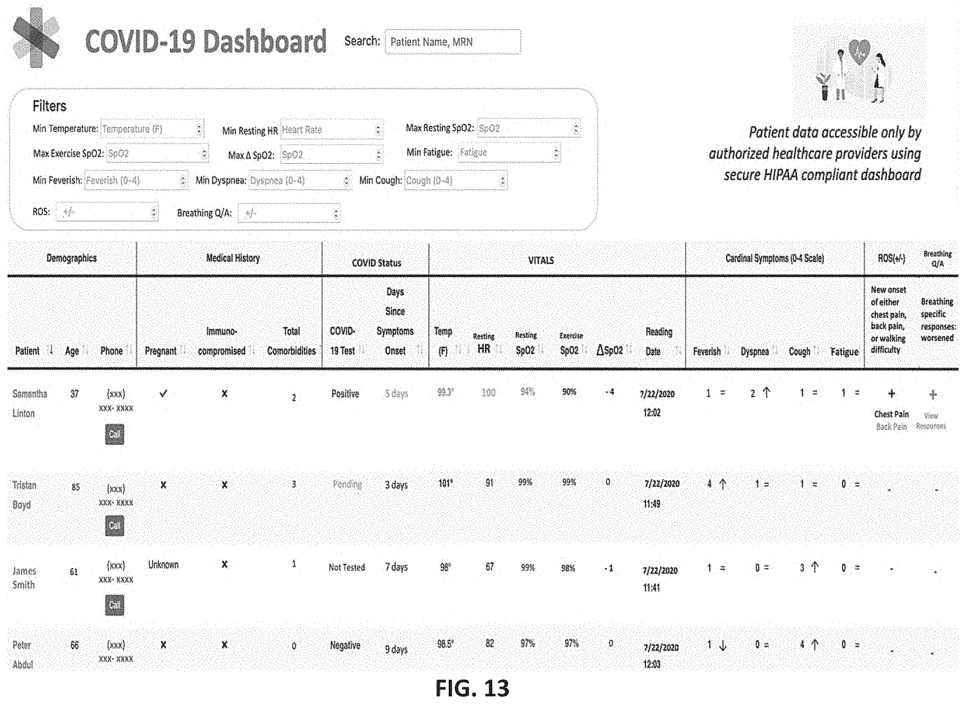

For example, as shown in FIG. 11A, patients may contact an automated phone system (e.g., by calling, texting, etc.) at regular intervals (e.g., daily, twice daily, etc.) to provide one or more bodily metrics for assessing the patient's condition. For example, a patient may be instructed to call into an automated, HIPAA-compliant, toll-free phone number for a daily assessment. In some variations, the automated phone system may be similar to that described in U.S. patent application Ser. Nos. 16/664,434 and/or 62/955,223 (incorporated by reference above). Furthermore, in some variations an automated, programmable text service may proactively reach out to a patient to prompt an assessment (e.g., send an SMS text to the patient with a link at one or more set times each day, such as “Please complete your assessment by calling XXX-XXX-XXXX.”). As shown in FIG. 11B, generally, through the automated phone system, the patient may be prompted to provide evaluations of one or more vital signs and/or one or more symptoms associated with the disease for which the patient is being monitored, testing results, etc.

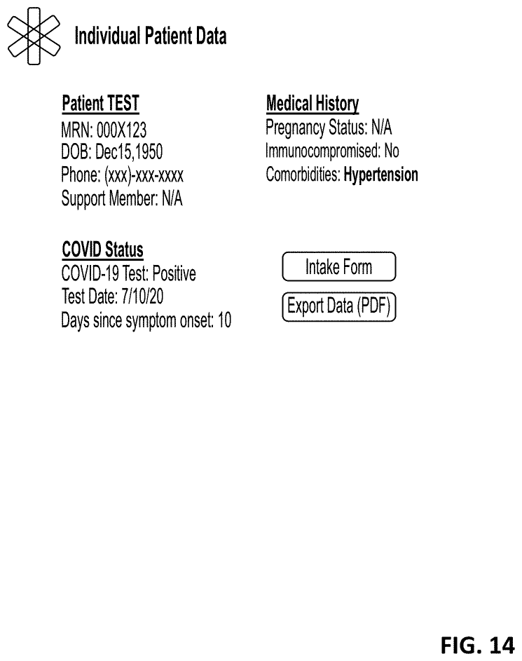

For example, through the automated phone system, the patient may be prompted to provide patient information, including one or more bodily metrics. FIG. 12 illustrates a set of exemplary prompts to be provided to the patient, in order to collect such information. For example, the automated phone system may prompt the patient to provide vitals (e.g., temperature, SpO₂, heart rate, etc.) and/or cardinal symptoms of the illness being monitored (e.g., fever, cough, shortness of breath, sore throat, fatigue for COVID-19) and their severity (e.g., on a numerical scale, such as between zero and four). Other aspects of the patient health may be assessed, such as nausea, vomiting, abdominal stomach pain, diarrhea, chills, muscle aches, runny nose, headache, sore throat, loss of smell and/or taste, etc. In some variations, the patient may be prompted using a set of dynamic questions which may, for example, be asked to the patient depending on the patient's responses to prior questions. For example, as shown in FIG. 12, the automated phone system may ask the patient about presence of muscle aches and chills if the patient reports having a fever of sufficient threshold severity (e.g., on a scale of zero to four, the threshold severity may be at least 1 in order to prompt the patient to report whether he or she is experiencing muscle aches and chills). As another example, if the patient reports having a cough of sufficient threshold severity, the automated phone system may follow-up by asking the patient whether he or she is experiencing difficulty associated with any mucus with cough/chest congestion, whether there is any blood in the cough/mucus, etc. As described in further detail below, other suitable dynamic questions may also be asked in follow-up to a provided response (e.g., the system may prompt certain questions to the patient conditioned upon certain previous responses by the patient).

Patient responses during the assessment are transcribed and processed by the remote monitoring system. For example, information that the patient has recited over a telephone call may be transcribed into text, parsed, and analyzed. Additional methods of analysis are described in further detail in U.S. patent application Ser. Nos. 16/664,434 and/or 62/955,223 (incorporated by reference above). It should be understood that similar to that described above, additional questions may be asked via the automated phone system to gather any changes or updates to other patient data, such as baseline medical risk information (e.g., new chronic medical conditions or medications, or indication that the patient is newly pregnant) and/or social exposure risk (e.g., recent visits to hospital, test results, close contact exposure to other person(s) at risk for the contagious disease, etc.). At least some information may additionally or alternatively be automatically pulled from the patient's medical record for consideration by the remote monitoring system (e.g., new positive test for the contagious disease).

In some variations, the provided patient information may be compared to protocols (e.g., CDC medical protocol) to trigger appropriate follow-up questions. The follow-up questions may be identified and asked in real-time or substantially real-time during completion of the patient assessment during completion of the online survey or phone assessment, and/or may be identified and/or asked after the completion of the online survey or phone assessment during a separate contact with the patient. Based on the patient information provided, disease progression may be identified at the patient level and triaged to appropriate personnel (e.g., heath system providers and county health officials).

### Patient Triaging

As described above, in addition to collecting vitals and symptoms regularly (e.g., daily, twice daily), a remote monitoring service may collect baseline information from patients at intake to help with risk stratification (e.g., patients who are elderly with chronic conditions and exposure history are at higher risk than younger patients without comorbidities) for triaging purposes.

In some variations, a triaging protocol may incorporate a “trending index” that may be generated to triage or rank all patients by potential medical risk. For example, a trending index can be reviewed and adopted by medical providers who are interested in remotely monitoring their patients at risk for the contagious disease of interest. In some variations, the trending index may be based at least in part on baseline medical risk, social exposure risk, and/or patient assessment (e.g., vital signs, symptoms). Additionally or alternatively, a triaging protocol may allow medical teams (or public health organizations) to sort patients based on characteristics such as age, comorbidity risk, social/exposure history, as well as symptoms/vitals and/or other suitable information.

In some variations, a triaging protocol may additionally or alternatively be based at least in part on a machine learning model (e.g., neural network algorithm) that has been trained using patient data obtained for patients who have been confirmed positive for the contagious disease. In other words, in addition or as an alternative to utilizing a trending index such as that described below, medical risk for patients may be predicted (e.g., by a learning module 138 as shown in FIG. 1B) based on historical information for known cases of the contagious disease.

As shown in Table 1, an exemplary trending index may incorporate multiple risk scores associated with different characteristics of the patient. For example, in some variations an exemplary trending index may be based on at least one risk score associated with each of multiple risk factor categories: baseline medical risk (e.g., age, chronic medical conditions), social exposure risk, at least one patient assessment (e.g., daily symptoms, vital signs), and/or days since first symptoms appeared. For example, a risk score may be based in part on raw points associated with individual aspects of baseline medical risk, social exposure risk, symptoms, vital signs, etc. In some variations, a risk score may be normalized for a risk factor category or subcategory based on a respective normalization value (e.g., normalization value of 15 for age). Additionally or alternatively, a risk score for a particular risk factor category may be capped at a respective maximum value as shown in the below Tables 2-5. The normalization value and the maximum value for a particular risk factor category may be equal. In some variations, a trending index may be the sum (or other suitable formula) of scores of risk scores for the risk factor categories. Furthermore, in some variations, the relative contribution of a risk factor category may be adjusted based on a respective weighting factor.

TABLE 1
Trending index score components

| | Raw Points | Normalized | Weighted | Total |
|---|---|---|---|---|
| Baseline Medical Risk: | Max 15 | /15 | x10 | |
| Age | | | | |
| Baseline Medical Risk: | Max 50 | /50 | x10 | |
| Chronic Conditions | | | | |
| Social Exposure History: | Max 30 | /30 | x10 | |
| Daily Symptoms + | Max 100 | /100 | x40 | |
| Vitals, Static | | | | |
| Daily Symptoms + | Max 100 | /100 | x20 | |
| Vitals, Dynamic | | | | |
| Days since onset of | Max 10 | /10 | x10 | |
| cardinal symptoms | | | | |

As an example variation, for monitoring COVID-19, baseline medical risk and daily symptoms/vitals factors may be individually scored as shown in Tables 2-7. However, it should be understood that in other variations (for COVID-19, for other contagious diseases, etc.), other suitable points and ranges may be used.

Table 2 illustrates example points associated with different age ranges, and Table 3 illustrates example points associated with different baseline medical risks. To the extent that either females or males may be at greater risk for a particular contagious disease, baseline risk points may similarly be associated with the patient's sex.

TABLE 2
Baseline risk stratification for age for COVID-19 monitoring

| Age | Points |
|---|---|
| >30 | +1 |
| >40 | +2 |
| >50 | +3 |
| >60 | +5 |
| >70 | +10 |
| >80 | +10 |

Age total points: _{——}_{——}

TABLE 3
Baseline risk stratification for chronic
conditions for COVID-19 monitoring

| “Chronic” Conditions/Meds | Points |
|---|---|
| Chronic Lung Disease: COPD (emph/c. bron) | +5 |
| Chronic Lung Disease: Asthma | +5 |
| Chronic Lung Disease: Other | +5 |
| Diabetes Mellitus | +3 |
| Cardiovascular disease (CHF) | +5 |
| Cardiovascular disease (ACS) | +5 |
| Cardiovascular disease (HTN) | +5 |
| Cardiovascular disease (arrhythmia) | +5 |
| Cardiovascular disease (other) | +3 |
| Chronic renal disease | +3 |
| Chronic liver disease | +3 |
| Immunocompromised condition | +5 |
| Neurologic/neurodevelopmental/intellectual disability | +3 |
| Obesity: BMI (kg/m²): 30-39 | +3 |
| Obesity: BMI (kg/m²): 40+ | +5 |
| Other Chronic Disease | +3 |
| Pregnant (if female) | +3 |
| Current Smoker | +3 |
| Past Smoker | +1 |
| Medication: ACE Inhibitor | +5 |
| Medication: ARB | +5 |
| Medication: Anti-inflammatory (NSAID/Cortisone) | |

Comorbidities/Meds total points (max out at 50): _{——}_{———}

Table 4 illustrates example points associated with different social exposure risk. In some variations, questions relating to recent foreign and/or domestic travel (marked with * in Table 4), may be customized based on factors such as the estimated incubation period, and/or estimated current degree of community spread, etc. of the particular contagious disease for which patients are being monitored. More points may, for example, be associated with regions associated with higher levels of travel notice (e.g., by the CDC) or based on otherwise known incidence of the disease. For example, recent travel to an epicenter region may be associated with +5 points, recent travel to a region with a Level 3 travel notice may be associated with +4 points, recent travel to a region with a Level 2 travel notice may be associated with +3 points, recent travel to another region known to have a developing outbreak may be associated with +2 points, etc. In some variations, questions relating to recent travel may be appropriate in instances where there is not yet known community transmission of the disease in a region where the patient is located. Such questions relating to recent travel may be omitted in instances where travel is not an important risk factor (e.g., in instances where there is known widespread community transmission of the contagious disease in a region where the patient in located).

TABLE 4
Risk stratification for social exposure history for COVID-19 monitoring

| Question | Points | (Live with) |
|---|---|---|
| Healthcare worker (or essential worker or other occupation in | +4 | +2 |
| high-risk environment) in the United States? | | |
| Healthcare worker (or essential worker or other occupation in | +4 | +2 |
| high-risk environment) outside the United States? | | |
| History of being in a healthcare facility (as a patient, worker or | +5 | +4 |
| visitor) in China? | | |
| *In the last X days: Travel to [region with high incidence of | variable | variable |
| contagious disease]? | | |
| Do you live in a nursing home or live in close proximity with | +2 | +1 |
| someone who works at a nursing home? | | |
| Have you been on a cruise ship recently or live in close proximity | +2 | +1 |
| with someone who has been on a cruise? | | |
| Have you been in a school dormitory? | +2 | +1 |
| Have you been in another close proximity situation? | +2 | +1 |
| Positive lab-confirmed COVID-19? | +30 | N/A |
| Pending COVID-19 test | +15 | N/A |
| Household contact with another lab-confirmed COVID-19 case- | +10 | N/A |
| patient? | | |
| Household contact with another suspected COVID-19 case-patient | +8 | N/A |
| [not lab confirmed]? | | |
| Community contact with another lab-confirmed COVID-19 case- | +10 | +5 |
| patient? | | |
| Community contact with another suspected COVID-19 case- | +8 | +4 |
| patient [not lab confirmed]? | | |
| Healthcare contact with another lab-confirmed COVID-19 case- | +10 | +5 |
| patient: [Patient, Visitor, Healthcare worker]: | | |
| Healthcare contact with another suspected COVID-19 case-patient | +8 | +4 |
| [not lab confirmed]: [Patient, Visitor, Healthcare worker]: | | |
| Contact with confirmed COVID-19 case in the US? | +10 | +5 |
| Contact with confirmed COVID-19 case from outside the US? | +10 | +5 |
| Exposure to animals? | +2 | +1 |
| Exposure to a cluster of patients with severe acute lower | +8 | +6 |
| respiratory distress of unknown etiology? | | |
| Other exposure: | +2 | N/A |
| Unknown: | +2 | N/A |

Social Exposure total points (max out at 30): _{——}_{——}

In some variations, the triaging protocol (e.g., trending index) may assess aspects of the regular (e.g., daily) patient assessment in a static manner (e.g., incorporating patient data from the most recent patient assessment), or in a dynamic manner (e.g., incorporating change in patient data relative to one or more previous patient assessments). Table 5 illustrates example points associated with vitals and symptoms from a patient assessment assessed in a static manner for COVID-19, while Table 6 illustrates example points associated with vitals and symptoms from a patient assessment assessed in a dynamic manner for COVID-19.

TABLE 5
Risk stratification for daily vitals/symptoms (static) for COVID-19

| Vitals | Points |
|---|---|
| Fever ≥ 100.4 F. (38 C.) | +10 |
| HR ≥ 120 | +12 |
| HR ≥ 110 | +9 |
| HR ≥ 100 | +6 |
| Fever ≥ 102.2 F. (39 C.) | +12 |
| Fever ≥ 104.0 F. (40 C.) | +14 |
| Resting % Spo2 ≤ 94 | +12 |
| Resting % Spo2 ≤ 92 | +14 |
| Resting % Spo2 ≤ 90 | +16 |
| Resting % Spo2 ≤ 88 | +18 |
| Resting % Spo2 ≤ 86 | +20 |
| Exercise % Spo2 ≤ 94 | +12 |
| Exercise % Spo2 ≤ 92 | +14 |
| Exercise % Spo2 ≤ 90 | +16 |
| Exercise % Spo2 ≤ 88 | +18 |
| Exercise % Spo2 ≤ 86 | +20 |
| Delta SpO2 (exercise-resting) ≤ −1 | +1 |
| Delta SpO2 (exercise -resting) ≤ −2 | +2 |
| Delta SpO2 (exercise -resting) ≤ −3 | +3 |
| Delta SpO2 (exercise -resting) ≤ −4 | +4 |
| Delta SpO2 (exercise -resting) ≤ −6 | +6 |
| Delta SpO2 (exercise -resting) ≤ −8 | +8 |
| Delta SpO2 (exercise -resting) ≤ −10 | +10 |
| | Weighted Points |
| Symptoms | |
| Subjective fever [Score 0-4] | Score x3 |
| Chills [Score 0-1] | Score x1 |
| Muscle aches (myalgia) [Score 0-1] | Score x2 |
| Cough (new onset or worsening of chronic | Score x3 |
| cough) [Score 0-4] | |
| Shortness of breath (dyspnea) [Score 0-4] | Score x6 |
| Runny nose (rhinorrhea) [Score 0-1] | Score x1 |
| Sore throat [Score 0-4] | Score x2 |
| Fatigue [Score 0-4] | Score x2 |
| Nausea or vomiting [Score 0-1] | Score x1 |
| Headache [Score 0-1] | Score x1 |
| Abdominal Pain [Score 0-1] | Score x1 |
| Diarrhea [Score 0-1] | Score x1 |
| Hemoptysis [Score 0-1] | Score x1 |
| Mucus/chest congestion [Score 0-1] | Score x1 |
| Loss of smell and/or taste | Score x1 |
| New Onset Symptoms | |
| New Onset Chest Pain | +20 |
| New Onset Back Pain | +10 |
| New Onset unsteady/walking difficulty | +10 |
| Other Symptoms | Points |
| Lacks satisfying breath | +10 |
| Out of breath walking/talking | +10 |
| Breathing worse lying down | +10 |
| Chest tightness/congestion | +10 |
| Can't catch breath | +10 |
| Breathing requires work/effort | +10 |
| Increased anxiety | +10 |

Vitals/Symptoms (static) total points (max out at 100): _{——}_{———}

TABLE 6
Risk stratification for daily vitals/symptoms
(dynamic) for COVID-19

| | Points |
|---|---|
| Change in Vitals | |
| Fever + 0.5 F. | +6 |
| Fever + 1.0 F. | +12 |
| Resting HR + 10 | +6 |
| Resting HR + 20 | +12 |
| Resting Sp02 drop 2-4 | +12 |
| Resting Sp02 drop 4+ | +24 |
| Exercise Sp02 drop 2-4 | +12 |
| Exercise Sp02 drop 4+ | +24 |
| Delta Sp02 (exercise-resting) drop 2-4 | +12 |
| Delta Sp02 (exercise-resting) drop 4+ | +24 |
| Symptom changes | |
| Feverish + 1 | +3 |
| Feverish + 2 | +6 |
| Feverish + 3 or more | +12 |
| Cough + 1 | +3 |
| Cough + 2 | +6 |
| Cough + 3 or more | +12 |
| Shortness of breath (dyspnea) + 1 | +6 |
| Shortness of breath (dyspnea) + 2 | +12 |
| Shortness of breath (dyspnea) + 3 or more | +24 |
| Sore throat + 2 | +1 |
| Sore throat + 3 or more | +3 |
| Fatigue + 2 | +1 |
| Fatigue + 3 or more | +3 |

Vitals/Symptoms (dynamic) total points (max out at 100): _{——}_{——}

Table 7 illustrates example points associated with days since first cardinal symptoms appeared for COVID-19. Accordingly, assessment of this risk category may incorporate patient data received from the patient over multiple patient assessments over time.

TABLE 7
Risk stratification for # days since
onset of cardinal symptoms of COVID-19

| Days since first cardinal symptoms | Points |
|---|---|
| 0-4 | 5 |
| 5-10 | 10 |
| 11-15 | 5 |
| 16-20+ | 1 |

Total points: _{——}

### Patient Data Communication and Alerts

In some variations, a group of patients (e.g., within a city, set of cities, county, state, etc., and/or within a group of patients associated with a healthcare institution or medical care provider) being monitored may be triaged based on their trending index scores. In some variations, patient data for a group of patients may be communicated via a dashboard or other suitable summary report interface, where the group of patients may be summarized (e.g., ranked) and/or information about an individual patient's health status may be accessed in an individual patient report. The communicated patient data displayed in the summary report (or communicated in any suitable manner) may include, for example, at least one patient assessment (e.g., symptoms, vital signs), testing status for the contagious disease, at least one trending index, and/or the results of triaging the one or more patients according to medical risk associated with the contagious disease.

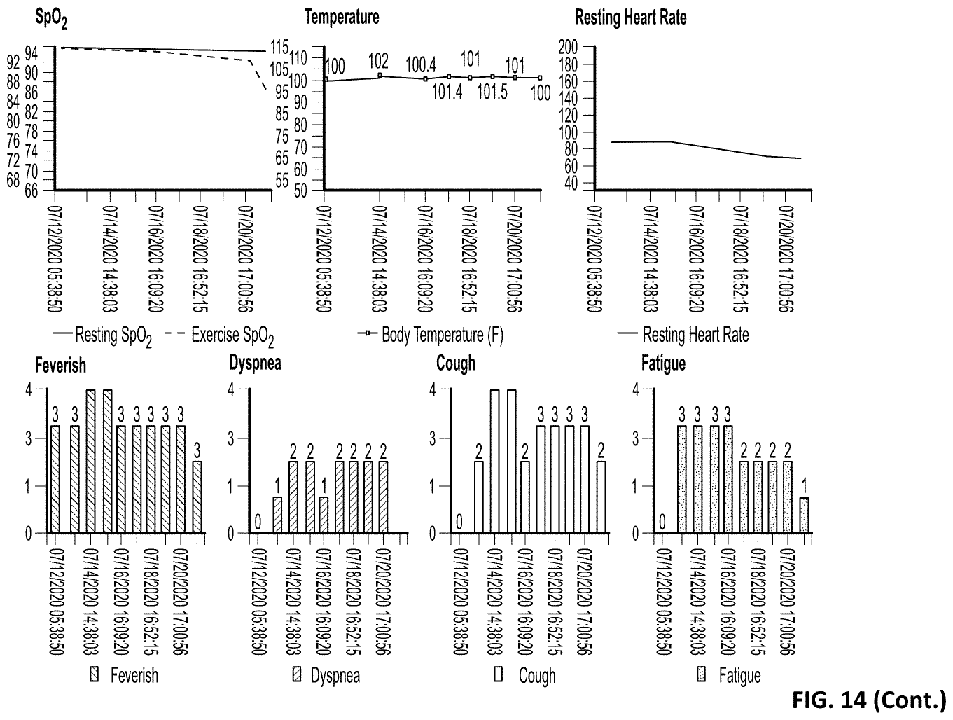

For example, FIG. 13 illustrates an exemplary graphical user interface (GUI) of a dashboard in which information for a group of remotely monitored patients may be displayed, such as demographics, medical history, infection status, vital measurements and/or trends, cardinal symptoms of the illness, and/or other suitable information. The patients may be rank-ordered by trending index score to help identify patients who are highest risk and require immediate medical attention. For example, certain patients may be elevated for medical attention (e.g., instructed to go to the hospital) and/or further action (e.g., follow-up telephone call with a medical care practitioner). Alerts may additionally and/or alternatively be sent to a monitoring medical care practitioner in response to a patient being flagged as sufficiently at-risk. Conversely, patients that are identified as lower risk may be instructed to continue self-monitoring at home or otherwise remotely. Information on the summary report may be color-coded or otherwise keyed for a reader to quickly review and assess the condition of the monitored patient. In some variations, alerts, thresholds, and other aspects of summary reports and patient reports such as those described in U.S. patent application Ser. Nos. 16/664,434 and/or 62/955,223 (incorporated by reference above) may also be incorporated in the methods described herein. Additionally or alternatively, patients presented on the dashboard or other summary report may be sorted any individual patient characteristic (e.g., risk category score, type of symptom, type of vital sign, status of test for the contagious disease, location, etc.).

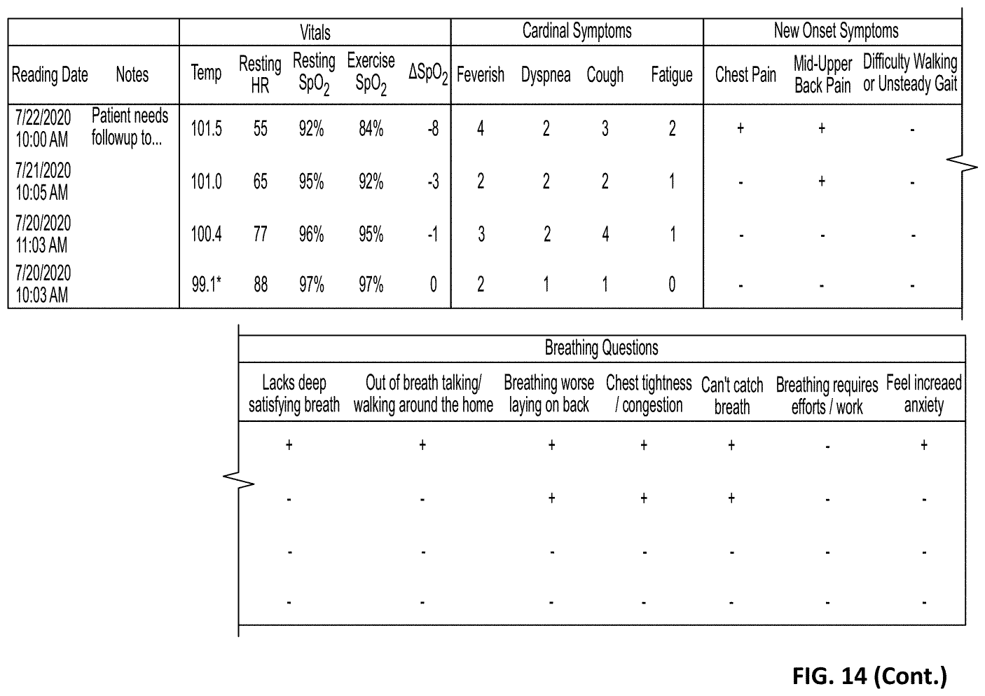

As described above in some variations, individual patients may be selected for more detailed review. For example, FIG. 14 illustrates an exemplary GUI of a patient report presenting more detailed information including temporal trends in vitals and/or symptoms, and/or any other information which may be useful to a monitoring medical care practitioner. In some variations, a individual patient report may be uploaded to the patient's medical record for reference. For example, the remote monitoring platform may be linked to an electronic health records system, and/or the generated individual patient report may be saved and exported appropriately for storage in the electronic health records system.

In some variations, the remote monitoring system may communicate a programmed alert for at least one patient characterized as having a threshold medical risk associated with the contagious disease (e.g., a threshold trending index value). The programmed alert may, for example, be communicated through emphasizing on the summary report (e.g., dashboard) of any patients who are characterized as having a threshold medical risk associated with the contagious disease. Such highlighting may appear as color-coding, enlarged or capitalized font, location of the patient listing (e.g., ranked position on a group list), etc. As another example, the programmed alert may be communicated by providing an alert notification (e.g., through a secondary dialog box, contact to the medical care provider such as via email, SMS text message, or phone call, etc.) for individual patients or a group of patients characterized as having threshold medical risk.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method for remotely monitoring one or more patients, the method comprising:
at one or more processors:
receiving a transmission of one or more patient assessments of current health of each patient, wherein each patient assessment comprises an evaluation of at least one of: at least one symptom known to be associated with a contagious disease and at least one vital sign associated with the contagious disease, wherein the contagious disease affects the respiratory system;
determining a trending index for each patient based on a baseline medical risk of the patient specific to the contagious disease, a social exposure risk of the patient, at least one patient assessment for the patient, and time since onset of the at least one symptom known to be associated with the contagious disease; and
triaging the one or more patients according to respiratory risk associated with the contagious disease based on the trending index; and
providing a respiratory treatment for at least one patient identified as having a threshold respiratory risk associated with the contagious disease.

2. The method of claim 1, wherein the contagious disease is a viral disease.

3. (canceled)

4. The method of claim 2, wherein the viral disease is COVID-19.

5. The method of claim 1, wherein the patient assessment comprises an evaluation of at least one symptom selected from the group consisting of: fever, cough, dyspnea, fatigue, chills, myalgia, rhinorrhea, sore throat, nausea, vomiting, headache, abdominal pain, diarrhea, hemoptysis, chest congestion, and loss of smell and/or taste.

6. The method of claim 5, wherein the evaluation of at least one symptom comprises a severity rating on a numerical scale.

7. The method of claim 1, wherein the patient assessment comprises an evaluation of at least one symptom selected from the group consisting of new onset chest pain, new onset back pain, and new onset walking difficulty.

8. The method of claim 1, wherein the patient assessment comprises an evaluation of breathing difficulty during one or more specified activities.

9. The method of claim 1, wherein the patient assessment comprises an evaluation of at least one vital sign selected from the group consisting of resting oxygen saturation, and oxygen saturation immediately following mild activity.

10. The method of claim 9, wherein the patient assessment comprises an evaluation of oxygen saturation measured at rest, and oxygen saturation measured immediately following mild activity.

11. The method of claim 10, wherein the patient assessment comprises an evaluation of the difference between oxygen saturation immediately following mild activity, and oxygen saturation measured at rest.

12. The method of claim 1, wherein the patient assessment comprises an evaluation of at least one vital sign selected from the group consisting of temperature and heart rate.

13. The method of claim 1, wherein determining the trending index for each patient comprises aggregating a first risk score associated with baseline medical risk for the patient, a second risk score associated with social exposure risk for the patient, and a third risk score associated with at least one patient assessment for the patient.

14. The method of claim 13, wherein the first risk score is quantified based on one or more characteristics selected from the group consisting of: age, sex, pregnancy, presence of a chronic medical condition, engagement in a chronic health-adverse behavior, and prescription of a specified medication.

15. The method of claim 13, wherein the second risk score is quantified based on one or more characteristics selected from the group consisting of: occupation in a high-risk environment, previous presence in a healthcare facility, previous presence in close proximity situation with other people, a positive test result for the contagious disease, a pending test result for the contagious disease, association with a person who is suspected or confirmed as tested positive for the contagious disease, and association with a person exhibiting symptoms of the contagious disease.

16. The method of claim 15, wherein the second risk score is further quantified based on the patient having close contact with a person having one or more characteristics selected from the group consisting of: occupation as a healthcare worker, previous presence in a healthcare facility, previous presence in close proximity situation with other people, a positive test for the contagious disease, a pending test result for the contagious disease, association with a person who is suspected or confirmed as tested positive for the contagious disease, and association with a person exhibiting symptoms of the contagious disease.

17. The method of claim 13, wherein the third risk score is quantified based at least in part on severity of a symptom or vital sign in a most recent patient assessment.

18. The method of claim 13, wherein the third risk score is quantified based at least in part on change in severity of a symptom or vital sign between the most recent patient assessment and at least one prior patient assessment.

19. The method of claim 13, wherein determining the trending index for each patient further comprises aggregating a fourth risk score associated with number of days since onset of the at least one symptom known to be associated with the contagious disease.

20. The method of claim 1, further comprising prompting each patient to complete the patient assessment.

21. The method of claim 20, wherein prompting each patient comprises communicating to at least one patient a SMS text message comprising a hyperlink to an interactive online survey.

22. The method of claim 20, wherein prompting each patient comprises communicating to at least one patient over an automated phone system with at least one of a programmable voice service and a programmable text service.

23. The method of claim 20, wherein prompting each patient comprises transmitting a programmed reminder to at least one patient to complete the patient assessment, in response to failing to receive the patient assessment from the at least one patient by a predetermined time.

24. The method of claim 20, further comprising transmitting a programmed reminder to a designated contact of at least one patient in response to failing to receive the patient assessment from the at least one patient by a predetermined time.

25. The method of claim 1, further comprising communicating to one or more medical care practitioners patient data associated with the one or more patients.

26. The method of claim 25, wherein the patient data comprises at least one selected from the group consisting of: at least one patient assessment, at least one trending index, and the results of triaging the one or more patients according to medical risk associated with the contagious disease.

27. The method of claim 25, wherein communicating to one or more medical practitioners patient data comprises communicating a programmed alert for at least one patient characterized as having a threshold medical risk associated with the contagious disease.

28. The method of claim 1, wherein at least one patient is awaiting results of a test for the contagious disease.

29. The method of claim 1, wherein at least one patient has tested positive for the contagious disease.

30. The method of claim 1, wherein at least one patient is associated with a person who has tested positive for the contagious disease.

31. A method for remotely monitoring one or more patients, the method comprising:
at one or more processors:
interfacing a remote monitoring system with an electronic medical record for one or more patients via an application programming interface (API);
receiving a transmission of one or more patient assessments of current health of each patient, wherein each patient assessment comprises an evaluation of at least one of: at least one symptom known to be associated with the contagious disease and at least one vital sign associated with the contagious disease;
determining a trending index for each patient based on a baseline medical risk of the patient specific to the contagious disease, a social exposure risk of the patient, at least one patient assessment for the patient, and time since onset of the at least one symptom known to be associated with the contagious disease;
triaging the one or more patients according to medical risk associated with the contagious disease based on the trending index; and
pulling or pushing patient data associated with each patient from or to the electronic medical record for the patient through the API.
